(19) United States
(12) Patent Application Publication
(10) Pub. No.: US 2010/0160903 A1
(43) Pub. Date: Jun. 24, 2010

(54) PROCESS AND SYSTEM FOR TREATING A VASCULAR OCCLUSION OR OTHER ENDOLUMINAL STRUCTURE

(76) Inventor: Yosef KRESPI, New York, NY (US)

Correspondence Address: IP Patent Docketing;K&L GATES LLP, 599 Lexington Avenue, 33rd Floor, New York, NY, 10022-6030, US

(21) Appl. No.: 12/642,192

(22) Filed: Dec. 18, 2009

Related U.S. Application Data
(60) Provisional application No. 61/139,879, filed on Dec. 22, 2008.

(51) Int. Cl. A61B 18/24; A61B 17/22

(52) U.S. Cl. 606/7; 606/128

(57) ABSTRACT

A process and instruments for diminishing an undesired endoluminal structure present at a treatment site in a mammalian treatment subject. The endoluminal can be or include a vascular occlusion, a biofilm or another undesired biological structure. The process can include applying mechanical shockwaves to the endoluminal structure and the endoluminal structure absorbing the applied mechanical shockwaves and becoming diminished, dispersed or weakened. The shockwaves can be generated by pulsed laser energy delivered to an ionizable target via an optical fiber.

### CROSS REFERENCE TO A RELATED APPLICATION

This application claims the benefit of provisional patent application No. 61/139,879, filed on Dec. 22, 2008, the entire disclosure of which is incorporated by reference herein.

### STATEMENT REGARDING FEDERALLY SPONSORED RESEARCH OR DEVELOPMENT

(Not applicable.)

The present invention relates to a process and system for diminishing or reducing a vascular occlusion or another undesired biological structure in the vasculature or other lumen of a mammalian treatment subject.

### BACKGROUND

U.S. 2008/0097251 to Babaev (“Babaev”) describes a method and apparatus for treating vascular obstructions which employs ultrasound in combination with cryogenic energy. As described by Babaev, “current methods” used to treat vascular obstructions put pressure on a blood vessel or deliver heat to the blood vessel resulting in stress on, or tissue damage to the a blood vessel.

Vascular obstructions and other mammalian endoluminal occlusions may be undesirable and may lead to possibly serious medical complications if not treated effectively. A simple and effective treatment process that can be applied to a variety of mammalian sites where endoluminal structures are present would be desirable.

### SUMMARY OF THE INVENTION

The present invention provides an endoluminal structure diminishment or reduction process which can reduce the mass of, eliminate, or weaken undesired endoluminal structures resident in or on mammals.

It has now been discovered that undesired endoluminal structures can be reduced by applying mechanical shockwaves or pressure pulses, or both, to the endoluminal structure. The shockwaves or pressure pulses can be generated using light energy, for example, light energy output by a laser, or by other suitable means.

In one aspect, the invention provides a process for diminishing an undesired endoluminal structure present at a treatment site in a mammalian treatment subject which comprises applying mechanical shockwaves to the endoluminal structure and the endoluminal structure absorbing the applied mechanical shockwaves and becoming diminished, dispersed or weakened. The endoluminal structure can be a vascular occlusion, a biofilm or another undesired biological structure. The endoluminal structure can be a structure obstructing a biological fluid flow path in the mammalian treatment subject, for example a blood vessel.

If the endoluminal structure is a biofilm, the biofilm may be secured to the treatment site by exopolysaccharide material and can comprise one or more microorganisms selected from the group consisting of bacteria, fungi, protozoa, archaea and algae.

The process can comprise delivering a shockwave generating device through a lumen of the mammalian treatment subject to address the treatment site. Diminishing the endoluminal structure can comprise reducing the mass of, disrupting, attenuating or destroying the endoluminal structure.

The effects of applying the mechanical shockwaves to the endoluminal structure can cause one or more pieces of the endoluminal structure to tear away from the residual endoluminal structure, or from the treatment site, and possibly can comprise oscillating the endoluminal structure.

The shockwaves or pressure pulses used for the treatment can be mechanical in nature and can be laser-generated, if desired, for example, by impinging a pulsed laser beam on an ionizable material, for example, a metal target, to generate a plasma. The process can form a- plasma adjacent to the ionizable target and can generate mechanical shockwaves emanating from the plasma and moving away from the ionizable target. The ionizable target can take any desired one of various forms. For example, the ionizable target can be metallic and can be a component of a treatment instrument, or can be a separate entity. Multiple ionizable targets can be employed in a treatment.

The mechanical shockwaves can be generated as non-convergent mechanical shockwaves and the process can include directing the non-convergent mechanical shockwaves on to the endoluminal structure resident at the treatment site.

In another aspect, the invention provides a treatment instrument for controlling an undesired endoluminal structure resident at a treatment site in or on a mammalian treatment subject. The treatment instrument can comprise a shockwave generating tip assembly configured to apply mechanical shockwaves to the treatment site to control and optionally diminish or weaken the endoluminal structure.

The tip assembly can comprise a short rigid portion connected to a flexible portion and can be configured for delivery to a treatment site through a curved lumen of the mammalian treatment subject.

Furthermore, the treatment instrument can comprise a flexible outer tube to extend from the treatment site to a location external to the mammalian treatment subject, a short rigid stabilizer tube located within the distal end of the outer tube and a tubular metal tip extending over the distal end of the outer tube and over the stabilizer tube.

Usefully, the tubular metal tip can comprise a distal nose and the distal nose can comprise an ionizable target for transducing laser energy into mechanical shockwaves. An optical fiber can extend along the treatment instrument and can have a distal end positioned adjacent the ionizable target. The optical fiber can be connectable with a pulsed laser energy source to receive pulses of laser energy from the laser energy source and discharge the pulses of laser energy from the distal end of the optical fiber to impinge on the ionizable target, outputting mechanical shockwaves.

The mechanical shockwaves can be output in a mechanical shockwave pattern extending distally and laterally of the treatment instrument to facilitate directing the mechanical shockwaves toward the treatment site.

The tip assembly comprises a self-supporting unit containable within a small circular cross-section accommodatable in, and moveable along a mammalian lumen to the treatment site.

If desired, the invention can comprise a process and system for performing a second antimicrobial step after application of shockwaves to a treatment site, for example as is described in patent application Ser. No. 12/642,021 of Yosef Krespi filed Dec. 18, 2009, attorney docket number 0525497.00023, the disclosure of which is herein incorporated by reference.

### BRIEF DESCRIPTION OF THE SEVERAL VIEWS OF THE DRAWING

Some embodiments of the invention, and of making and using the invention, as well as the best mode contemplated of carrying out the invention, are described in detail herein and, by way of example, with reference to the accompanying drawings, in which like reference characters designate like elements throughout the several views, and in which:

FIG. 1 is a sectional view on the line 1-1 of FIG. 2 of a tip assembly for a mechanical shockwave treatment instrument suitable for endoluminal use, according to one embodiment of the invention;

FIG. 2 is a front view of the tip assembly shown in section in FIG. 1;

FIG. 3 is a sectional view on the line 3-3 of FIG. 5;

FIG. 4 is an opposite side elevation of the metal tip shown in FIG. 3;

FIG. 5 is a front view of the metal tip shown in FIG. 3 of a metal tip, the metal tip being a component of the tip assembly shown in FIG. 1;

FIG. 6 is a perspective view of a cylindrical support sleeve, the cylindrical support sleeve being a component of the tip assembly shown in FIG. 1;

FIG. 7 is a sectional view on the line 7-7 of FIG. 8 of a tip assembly for a mechanical shockwave treatment instrument suitable for endoluminal use, according to another embodiment of the invention;

FIG. 8 is a front view of the tip assembly shown in section in FIG. 7; and

FIG. 9 is a perspective view of a grooved support sleeve, the grooved support sleeve being a component of the tip assembly shown in FIG. 6.

### DETAILED DESCRIPTION OF THE INVENTION

The processes of the invention described herein usefully can be employed in the treatment of mammals, including in particular, humans. In addition, these processed can be applied to treatment of non-human mammals including, for example, horses, cattle, sheep, llamas, husbanded animals, pets including dogs and cats, laboratory animals, for example, mice, rats and primates, animals employed for sports, breeding, entertainment, law enforcement, draft usage, zoological or other purposes.

Processes according to the invention can be practiced to diminish, reduce or control an undesired endoluminal structure or occlusion resident at any one of a number of non ophthalmic treatment sites in a mammalian subject. For example, the treatment site selected from the group consisting of a coronary artery, a peripheral artery, an otolaryngological site, a nasal, sinus, or middle ear cavity, an implant site, a cardiac implant site, an endovascular implant site, a shunt, an orthopedic implant site, a gynecological implant site, an intrauterine device site, an urologic implant site and a urinary catheter site.

Treatment processes according to the invention for treating undesired endoluminal structures can provide complete or partial elimination of, attrition of, recanalization of, or other desired control of, a endoluminal structure resident in or on a treatment subject mammal, in particular, a human being. In some cases, the shockwave treatment may disrupt the integrity of the luminal obstruction, or weaken the obstruction, or loosen the adhesion of the obstruction to its supporting tissue or other structure, without removing material from the obstruction. Such disruption, weakening or loosening may allow or promote natural events to effect a reduction in the obstruction over time.

In some cases a single treatment can be effective to provide adequate reduction of the endoluminal structure. Multiple passes may be employed in the course of a single treatment. The invention also includes processes wherein an endoluminal occlusion is treated repeatedly at intervals, for example, of from about four hours to about a month. The treatments can, if desired be repeated at intervals of from about 1 to about 14 days.

Processes according to the invention can employ any suitable treatment instrument which can apply shockwaves, pressure pulses or other suitable non-chemical mechanical or energetic forces to mammalian endoluminal occlusions to diminish or destroy them, without unacceptable damage to subject tissue, for example, so that the tissue at the treatment site remains intact. The mechanical shockwaves can be generated by laser or other photic means, piezoelectrically or in another desired manner.

U.S. patent application Ser. No. 12/139,295 (Attorney Docket No. 0525497.00011), the disclosure of which is incorporated by reference herein, describes and claims a process for treating biofilms wherein shockwaves are applied to a biofilm to disperse it. In vitro data described in that application demonstrate a shockwave treatment causing a biofilm to oscillate, tearing and disintegrating the biofilm and substantially removing the biofilm from a site of attachment such as a bundle of sutures, an orthopedic screw or a tympanostomy tube. Some of the processes and devices disclosed in application Ser. No. 12/139,295 can be employed for the purposes of the present invention, as will be, or become, apparent to a person of ordinary skill in the art.

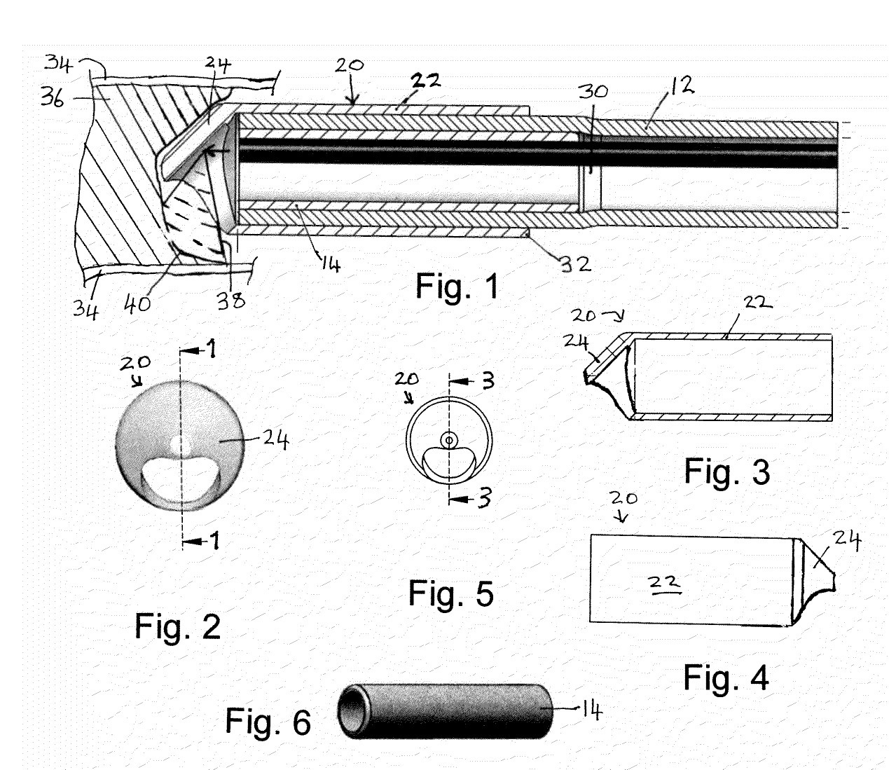

The tip assembly illustrated in FIGS. 1-6 can be employed in conjunction with a suitable delivery system, for example a catheter or an endoscope or the like, to access and apply shockwaves to endoluminal structures located at treatment sites located in a lumen of a subject mammal being treated, for example a vascular, bronchial, urinary, biliary, fallopian or seminal passage, tube or duct.

Referring to FIGS. 1-6, the illustrated tip assembly, referenced 10, comprises a shockwave generating device which can be delivered to address an internal treatment site in a human subject or other mammal, for example by employing an introducer. Tip assembly 10 comprises a flexible outer tube 12 which extends proximally (to the right as viewed in FIG. 1) back to control and supply systems (not shown) for the tip assembly 10, which control and supply systems will usually be located externally of the treatment subject.

A short, rigid stabilizer tube 14 is fitted tightly within the distal (lefthand in FIG. 1) end of outer tube 12. As shown, outer tube 12 is radially stretched, and the girth of outer tube 12 is expanded where it fits over stabilizer tube 14, thereby securely joining the two components together. Stabilizer tube 14 can help give structural integrity to tip assembly 10 and, with adequate length can facilitate manipulation and positioning of tip assembly 10 at an internal mammalian treatment site. However, for some applications, for example where the tip assembly 10 must traverse one or more turns or bends in a lumen to reach the target site, excess length may be undesirable. Accordingly, for such applications stabilizer tube 14 can have a length in the range of from about 4 mm to about 18 mm or to about 12 mm, for example a length in the range of from about 4 mm to about 7 mm. In one specific example, stabilizer tube 14 has a length of about 5.5 mm and an outer diameter of about 1.3 mm. In other cases, stabilizer tube 14 can have any suitable length as will be, or become, apparent to a person of ordinary skill in the art.

An optical fiber 16 is supported within stabilizer tube 14 and outer tube 12 and extends proximally to a light source (not shown). Optical fiber 16 can be secured to stabilizer tube 14, and if desired to outer tube 12, by any suitable means, for example adhesive, clips, clamps or the like and is connected externally of the treatment subject to a suitable pulsed laser light source (not shown).

Distally, outer tube 12, stabilizer tube 14 and optical fiber 16 terminate flush with a disk-like end plate 18 which extends across the end of outer tube 12 to closes distal end of outer tube 12 against ingress of debris, dislodged material, fluids and foreign materials that could clog tip assembly 10 or otherwise impede its operation.

A metal tip 20 is fitted over the distal end of outer tube 12. Metal tip 20 comprises a tubular shank 22 and a forwardly projecting, nose 24. If desired, the proximal end 26 of shank 22 of metal tip 20 can be crimped onto outer tube 12 supported by stabilizer tube 14. As can be seen from FIGS. 2 and 5, when read with FIG. 1, nose 24 of metal tip 20 can have a generally conical shape, with an output port 26 cut out of the conical wall. Desirably, the apex of the conical shape of nose 24, the point of the nose, is rounded, or otherwise smoothly contoured and is centrally located on the cross-section of tip assembly 10, lying approximately on the axis of metal tip 20 and tip assembly 10. These features of nose 24 can facilitate control of the manipulation of tip assembly 10 to advance through a lumen of a mammalian subject and to address a target site, discouraging lateral thrusts into lumen sidewall tissue, or “digging”, which may occur with asymmetric or sharp-edged nose configurations.

Output port can extend over any desired portion of the circumference of the cone, for example a portion of from about 10 to about 50 percent of the circumference. In the example shown in FIG. 2, output port 26 extends around about 20-40 percent of the circumference of nose 24.

Nose 24 is hollow with an internally curved, part conical target surface 28 on which a laser beam emerging from optical fiber 16 can impinge. As shown, nose 24 desirably overlies the distal end face of optical fiber 16 to act as a shield preventing high energy laser light from emerging from tip assembly 10 and directly impinging on sensitive tissue or other sensitive structure. Desirably, the point of nose 24 is smoothly contoured, for example rounded and free of sharp contours, to facilitate delivery through the subjects vascular system or other lumen or into a bodily cavity.

The various described tubular components of tip assembly 10, including outer tube 12, stabilizer tube 14 and shank 22, can have a circular cross-sectional shape, or another cross-sectional shape, if desired, for example, oval, elliptical, triangular, polygonal or the like. So long as they can cooperate together for the purposes of the present invention, the various tubular components can have different cross-sectional shapes, one from the other, if desired.

Suitable materials for the various components of tip assembly 10 which will enable tip assembly 10 to function in accordance with the objectives of the invention will be, or become, apparent to a person of ordinary skill in the art in light of this disclosure. For example, outer tube 12 can be formed of a durable flexible and protective material, such for example as a silicone polymer. Stabilizer tube 14 desirably is formed of a relatively rigid, load-bearing material for example stainless steel. Optical fiber 16 can be any suitably light-transmissive fiber of glass or the like which has sufficient flexibility for delivery to the treatment site. End plate 18 can be formed of a suitable material having good light transmissivity for the wavelength employed, for example glass or a suitable transparent plastic. Metal tip 20 can be formed in one piece of an ionizable metal suitable for generating shockwaves, for example titanium, stainless steel or zirconium. Alternatively, metal tip 20 can be formed of another material, possibly a nonmetallic material and can be provided with an ionizable metal insert to serve as target surface 28.

Outer tube 12 has a zone of flexure 30 adjacent to the proximal end 32 of stabilizer tube 14 where outer tube 12 is stretched to fit over stabilizer tube 14. Zone of flexure 30 helps give the treatment instrument flexibility upstream of the short rigid tip assembly 10, so as to help adapt the instrument for catheter delivery. Combined with tip assembly 10 having only a short length of inflexibility, as is further described herein and outer tube 12 and optical fiber 16 both being flexible, the instrument can be fabricated to be suitable for snaking or threading through the vasculature or other lumen or lumens, to a treatment site in the subject mammal. Less flexible instruments can be provided for treating other sites, if desired.

Tip assembly 10 is shown inserted into a vascular lumen 34 of a subject mammal, at a treatment site where vascular lumen 32 is blocked by a vascular occlusion 36. As shown, vascular occlusion 36 completely plugs or blocks vascular lumen 36 and its frontal surface, as presented to tip assembly 10, has been partially removed by shockwave treatment pursuant to the invention. Alternatively, vascular occlusion 36 could partially block vascular lumen 34. For example, vascular occlusion 36 could comprise a layer of plaque significantly constricting the fluid flow passage provided by vascular lumen 34. Vascular occlusion 36 can be constituted by a variety of materials, for example, plaque an embolic material, fibroid tissue and the like or a biofilm or other structural assemblage of foreign, possibly pathogenic microorganisms. Nose 24 of tip assembly 10 protrudes into vascular occlusion 36 and is removing material therefrom, as is further described below.

Tip assembly 10 can have any desired size and configuration according to its intended purpose as will be, or become, apparent to a person of ordinary skill in the art in light of this disclosure. For example, tip assembly 10 can be configured for delivery to internal mammalian body treatment sites. Delivery can be effected with the assistance of a catheter, or other introducer, to introduce tip assembly 10 into the body of a subject to be treated in a minimally invasive manner, for example subcutaneously.

To facilitate delivery, tip assembly 10 can be a small, self-supporting unit, free of projections or sharp edges, which is containable within a small circular cross-section suitable for accommodation in and movement along a mammalian lumen, for example an artery. Pursuant to one aspect of the invention, tip assembly 10 can fit within a containing cross-sectional circle which is less than about 5 mm in diameter, for example in a range of from about 2 to about 3 mm in diameter, or smaller. For example, the outer diameter of metal tip 20 can be about 2.5 mm or about 2.1 mm.

Also, to facilitate movement of tip assembly 10 around bends or curves in the subject mammal's vasculature or other lumen, tip assembly can be free of long rigid components, referring to the distal-proximal direction of introduction. Usefully, the longest rigid, or non-flexible, extent of tip assembly 10 can be less than 20 mm. According to the desired application the longest rigid, or non-flexible, extent of tip assembly 10 can be less than 15 mm, less than 10 mm or less than 8 mm. For example the metal tip 20 and stabilizer tube 14 can together provide a relatively inflexible structure having a length, from nose 24 to proximal end 32 of stabilizer tube 14, of about 7 mm.

Desirably, to reduce its size and to help fit within a useful containment circle, tip assembly 10 can be free of components commonly associated with a handheld instrument, such as a hand grip or hand piece and mechanical controls such as switches or valves. Also, tip assembly 10 can be free of conduits for bringing to the treatment site services such as irrigation, aspiration, illumination and inspection. Such services, when required can be provided by ancillary equipment such as an introducer or endoscope, as is further described herein.

In a further aspect, the present invention provides a treatment system comprising tip assembly 10 and a pulsed laser light source (not shown), with associated controls, disposed externally of the mammalian treatment subject, wherein the tip assembly 10 is functionally coupled to the laser light source to receive light therefrom by optical fiber 16.

Useful ancillary services can, in some instances be provided in parallel with, or alongside, the delivery of shockwaves via tip assembly 10. However, where space is confined at the treatment site, for example in a narrow vessel or lumen, irrigation, aspiration, illumination and/or inspection, or other useful services can be delivered to the treatment site, in series, or sequentially with the shockwave treatment, optionally in repeated cycles. For example, a treatment cycle can comprise illumination and inspection, followed by shockwave treatment followed by irrigation and/or aspiration to remove debris and the cycle can then be repeated, if desired.

Some examples of suitable systems and devices comprising introducers, endoscopes and/or apparatus for providing ancillary services such as irrigation, aspiration, illumination and/or inspection and the like, which can be employed in practicing the present invention, with suitable modification as will be, or become, apparent to a person of ordinary skill in the art, are disclosed in international publications Nos. WO 2008/124,376, WO 2008/124,376 and WO 2008/124,376, to Medtronic Xomed, Inc. The disclosures of said three international publications are herein incorporated by reference. The invention includes such systems and devices suitably modified to include shockwave application means as described herein.

In use, tip assembly 10 is advanced to the treatment site through the vasculature employing the assistance of a catheter, an introducer or other suitable delivery device, as described herein. The user manipulates the introducer to position tip assembly in juxtaposition to a desired treatment site. Manipulation can include snaking tip assembly 10 through an elongated bodily lumen, for example an artery, and may entail negotiating one or more bends, intersections or turning points or the like, requiring flexibility in the introduced instrumentation. Manipulation and positioning of tip assembly 10 can be monitored with an endoscope, if desired. For convenience, the endoscope, or endoscopic system can include and imaging system and can provide real time imaging of the environs of tip assembly 10 on a display screen visible to the user. When properly positioned, the laser light source is activated to effect the shockwave treatment and to transmit along optical fiber 16 a stream of laser pulses having suitable energy and timing parameters, as described herein.

Laser energy pulses emerge from the distal end face of optical fiber 16, through end plate 18 and strike target surface 28 which ionizes, creating a plasma and generating shockwaves. The shockwaves emanate from target surface 28 in the vicinity of the point of impact on target surface 28 of the light pulses leaving optical fiber 16, as shown by the arrow. The shockwaves radiate from this point of impact and are guided by the configuration of target surface 28 and output port 26 to leave tip assembly 10 in a divergent shockwave beam 38 and strike the treatment object 40. In the example shown, treatment object 40 is a portion of vascular occlusion 36.

The pattern of shockwave beam 38 is determined to a significant extent by the geometry of nose 24 and output port 26 and in the example shown the pattern is constrained to occupy only a small proportion of the spherical volume centered on the laser beam point of impact. Thus the shockwave energy is directed and concentrated into a limited volume, and can impact treatment object 40 over a limited area, but is not focused to a point or similarly small area. The received energy concentration at the surface of treatment object 40 varies substantially with the distance of output port 26 from treatment object 40. Accordingly, subject to the geometry of the anatomy at the treatment site, the user can vary the energy density by manipulating the treatment distance. Nose 24 can effectively prevent shockwaves leaving tip assembly 10 other than through output port 26 or in directions opposite to the direction of output port 26 from the point or zone of origin of the shockwave.

The angular spread of shockwave beam 38, as measured from its point (or zone) of origin at the point of impact of the laser beam on target surface 28 desirably is substantially less than 180°, in its major dimension and can, for example, be 150° or less, or 120° or less. If shockwave beam 38 is not approximately conical or square sectioned and therefore has a minor angular dimension, the minor angular dimension can be 120° or 90° less.

Surprisingly, by generating shockwaves having a high intensity and a short duration, the present invention makes it possibly to apply sufficiently strong forces to biological materials such as may constitute endoluminal occlusions without generating sufficient heat to cause tissue damage in sensitive anatomical environments. Surprisingly also, short-lived shockwave pulses, of duration and intensity, such as is described herein, or is implied by the parameters of the applied laser light pulses, can effectively disrupt or dislodge biological material impacted at the target site, notwithstanding the short duration of the shockwaves. Also, it is believed the shockwaves will travel effectively through water, aqueous liquids, serum, blood and other biological fluids to encounter and be absorbed by solid structures, in their path such as treatment object 40.

The shockwave treatment can be conducted in a progressive manner to incrementally, reduce, erode, ablate, abrade or otherwise removal material from, disrupt, disperse and/or weaken vascular occlusion 36. The user can rotate, reciprocate, translate, advance and retract, or otherwise manipulate tip assembly 10 as appears appropriate to weaken, reduce or destroy vascular occlusion 36. The treatment can be continued to open a passage through the occlusion or to remove it or to another desired completion point. If desired, as described herein, irrigation, aspiration, inspection, illumination or other adjunctive services can be performed concomitantly with the shockwave treatment or intermittently, in between steps of shockwave treatment.

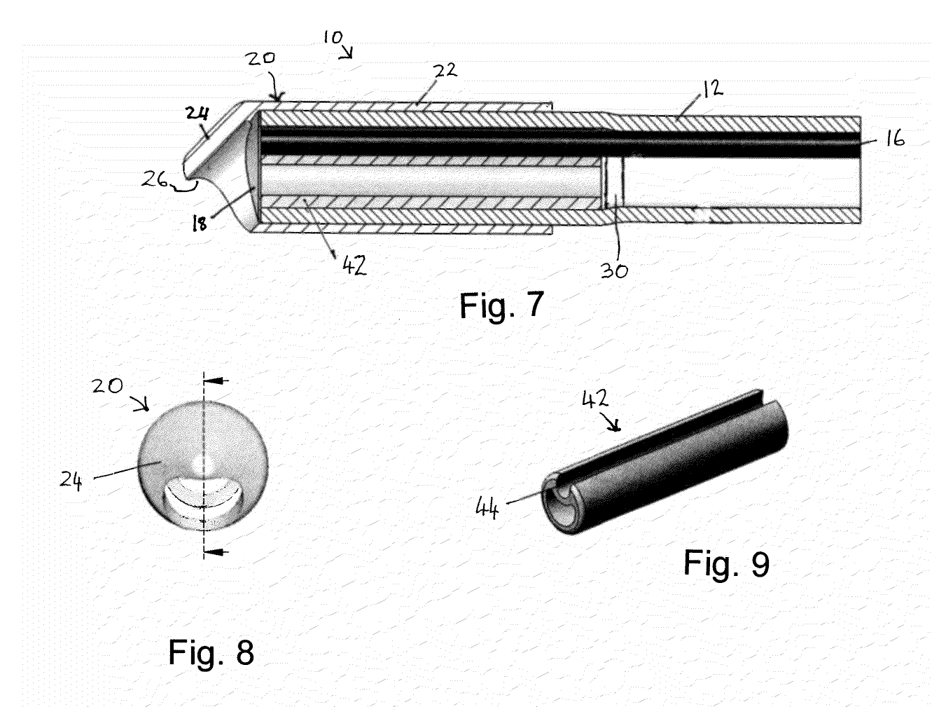

FIGS. 7-9 show a modified embodiment of tip assembly 10 which is generally similar to the embodiment shown in FIGS. 1-6 with the difference that a plastic sleeve 42 replaces stabilizer tube 14. Plastic sleeve 42 is configured with a groove 44 on its upper side, as viewed in FIGS. 7-9 which groove is a close fit around optical fiber 16. If desired plastic sleeve 42 can be formed, for example by molding, from a resilient, relatively rigid synthetic polymer, such as a polycarbonate polymer. Optical fiber 16 can snap into groove 44 or be threaded in lengthwise, if desired. Plastic sleeve 42 can support and positively locate optical fiber 16 in position within outer sleeve 12 without use of adhesive, clamps or the like.

Some examples of shockwave-generating surgical instruments, which with appropriate modification can be employed in the practice of the present invention are disclosed in Dodick et al. U.S. Pat. Nos. 5,906,611 and 5,324,282 (referenced jointly as “Dodick et al.” herein). The disclosure of each of the Dodick et al. patents is incorporated by reference herein. Some uses and modifications of the Dodick et al. instrument are disclosed in Thyzel U.S. Patent Application Publication No. 2007/0043340 (referenced as “Thyzel” herein).

The instruments described by Dodick et al. are useful for eye surgery for and particularly for cataract removal. As described by Dodick et al., the Dodick instrument is a laser-powered surgical instrument that employs a target for transducing laser energy into shockwaves. The instrument can be used in eye surgery, particularly for cataract removal. The Dodick instrument can comprise a handpiece holding a surgical needle and an optical fiber extending through a passageway in the needle. An open distal aspiration port for holding tissue to be treated communicates with the passageway through the needle. An optical fiber can extend along the length of the needle and have its distal end positioned close to a metal target supported by the instrument. Also as described by Dodick et al., pulses of laser energy are discharged from the distal end of the optical fiber to strike the target. The target, which can be formed of titanium metal, is described as acting as a transducer converting the electromagnetic energy to shockwaves that can be directed onto tissue in an operating zone adjacent to the aspiration port. If desired, the needle can be flexible to enhance access to treatment sites.

Some embodiments of the present invention can employ the shockwaves generated at the instrument's distal port, to impinge on and destroy, or attenuate, a subject-resident endoluminal structure attached to subject tissue, to an implant surface or to another treatment surface located in the operating zone adjacent the treatment instrument's distal port. The process can be performed with or without aspiration through the treatment instrument's distal port or through another port in the treatment instrument or another device.

Also, the treatment processes of the invention can be controlled to be non-damaging to subject tissue or to cause only modest, acceptable damage compatible with the seriousness of the infection. This is unlike the process described by Dodick et al. which comprises the fracturing of tissue.

The laser energy pulses employed to induce the shockwaves or pressure pulses used in the endoluminal structure treatment processes of the invention can be provided by any suitable laser. For example, as described by Dodick et al., a neodymium-YAG laser providing light energy at a wavelength of 1,064 nanometers with a pulse width of approximately 8 nanoseconds can be employed. Alternatively, other laser types can be employed, for example, gas lasers or solid lasers.

The laser energy pulses can be provided with any suitable repetition rate, for example, a pulse rate of from about 0.5 Hz to about 200 Hz, a pulse rate of from about 2 Hz to about 50 Hz or a pulse rate of from about 2 Hz to about 6 Hz. Pulse rates up to 100 or 200 Hz can be employed, if desired.

Any suitable pulse energy can be employed, for example, in a range of from about 2 millijoules (“mJ” herein) to about 15 mJ of energy per pulse or from about 6 mJ to about 12 mJ per pulse.

The energy pulses can have any suitable pulse width, for example from about 2 nanoseconds (“ns” herein) to about 20 ns or from about 8 ns to about 12 ns.

Some embodiments of the invention can employ a pulse duration of from about 8 to about 12 nanoseconds, a pulse rate of from about 2 to about 6 pulses per second or Hz and an energy per pulse of from about 6 mJ to about 10 mJ or 12 mJ.

In some cases, utilizing suitable energy parameters, from about 200 to about 800 shockwave-generating laser energy pulses can be employed to treat a endoluminal structure or a portion of a endoluminal structure at the distal port of the treatment instrument or in the vicinity of another ionizable target structure or structures.

Employing an optical fiber to deliver the laser energy, any suitable fiber-to-ionizable-target distance can be employed, for example, from about 0.7 mm to about 1.5 mm.

For treatment of cardiac, orthopedic, gynecologic, urologic or other implants, the treatment instrument can be adapted for catheter delivery of the distal tip of the treatment instrument to a treatment site via a suitable blood vessel or vessels, for example, an artery. Alternatively, the treatment instrument can be appropriately modified for subcutaneous delivery, for example, for laparoscopic delivery. The invention includes endoluminal structure treatment processes wherein the treatment instrument is delivered via a catheter, or laparoscopically, or in other suitable manner.

In some embodiments of the invention, the treatment instrument can comprise an inspection fiber to view the treatment site and monitor the progress of the treatment. This capability can be useful for treatment sites which are unexposed or concealed including internal sites such as the upper nose and sinuses and implant surfaces. The inspection fiber can have a distal input end disposable in the vicinity of the applicator needle tip to survey the treatment site and a proximal output end communicating optically with an output device viewable by a surgeon or other operator performing the treatment. The output device can be a video screen, an optic member, or another viewing element. If desired, the inspection fiber can extend through or alongside the treatment instrument or can comprise a separate device. The inspection fiber can enable the operator to monitor the treatment and manipulate the treatment instrument accordingly.

Dodick et al. U.S. Pat. No. 5,324,282 describes a flexible needle employing aspiration through the needle wherein shockwaves are reflected to a tissue-receiving zone inside the instrument. No stabilizing structure for the needle is described, nor is the “nose” configured as described herein. Thyzel also describe integrating the tip of the laser hand piece (treatment instrument) along with an optical fiber into a flexible endoscope and additionally employing optical imaging to enable treated sites to be visually monitored. However, no corresponding structure is described.

In some embodiments of the processes of the present invention, one or more of a number of treatment parameters to facilitate or improve performance of the treatment can be adjusted and improved or optimized for a particular application, for example by manipulation of an appropriate control, or instrument or other device by the surgeon or other operator. These parameters include the orientation, location and/or disposition of the treatment instrument, the application of saline or other irrigation fluid, the application of suction, and any one or more of the energy parameters employed to generate the applied pressure pulses. The energy parameters include the intensity, frequency, and pulse duration of the pressure pulses.

In the treatment of concealed treatment sites, adjustment of the treatment parameters can be facilitated by providing illumination means at the treatment site to illuminate the treatment site, as described herein. This measure can permit the surgeon, or other operator, to adjust one or more of the treatment parameters according to what he or she sees at the treatment site. Accordingly, some embodiments of the invention comprise illuminating the treatment site.

A further embodiment of treatment instrument according to the invention comprises illumination means or an illumination device to illuminate the target area to facilitate monitoring of the treatment. If desired, the illumination means can comprise an illumination fiber having proximal light input end communicating with a light source and having a distal light output end locatable in the vicinity of the treatment site to illuminate the treatment site. The illumination fiber can be movable with the treatment instrument. For example it may be a component of the treatment instrument or it can be a separate device. Illumination means not only can be usefully employed to illuminate concealed treatment sites but may also be useful for treatment of endoluminal structures resident at exposed treatment sites.

A still further process embodiment of the invention comprises impinging pulsed laser energy from an optical fiber on to an ionizable target, wherein the target comprises a metallic structure or material supported independently from the optical fiber.

The metallic structure can comprise one or more particles or pieces of a suitable metal, for example, tantalum. Other suitable metals, for example, titanium, stainless steel or zirconium can be employed, if desired. Metal powder can be employed, for example, a metal powder having an average particle size in the range of from about 0.1 micron to 100 micron. One useful metal powder is a tantalum powder having an average particle size of from about 1 micron to 5 micron available under product code TA 101 from Atlantic Equipment Engineers, Bergenfield, N.J.

The metal powder or possibly one or more small pieces of metal can be located in the vicinity of the target endoluminal structure, for example on one or more surfaces of the endoluminal structure, or nearby. Pulses of laser energy, can then be directed at the metal powder from the end face or bared tip of an appropriately located optical fiber.

In such embodiments of the invention, the ionizable target can be separated from the optical fiber. Thus, the laser energy can be delivered by an essentially bare, or lightly protected optical fiber. For example, the optical fiber can merely bear a thin fiber coating or protective sleeve or the like. Such an optical fiber lacking the encumbrance of a target bearing instrument housing or tubing can be relatively introduced into small, possibly remote, vasculature or other lumens employing a suitable catheter or other introducing device or means. If desired, the target metal powder or other separate metal target or targets can be introduced to the target treatment site separately from the optical fiber, for example, beforehand. If desired, the metal target powder or the like can be introduced through the same catheter or other introducer as the optical fiber.

Other pressure pulse generators that can be employed in the practice of the present invention include piezoelectric, for example piezoceramic, devices, spark discharge devices, electromagnetically or inductively driven membrane pressure shock wave generators or pressure pulse generators and generators that employ pressure currents or jets associated with the transport of material. The pressure pulse generator can be disposed in the treatment instrument or externally in a separate unit connected to the treatment. instrument by a transmission line.

The foregoing detailed description is to be read in light of and in combination with the preceding background and invention summary descriptions wherein partial or complete information regarding the best mode of practicing the invention, or regarding modifications, alternatives or useful embodiments of the invention may also be set forth or suggested, as will be apparent to one skilled in the art. The description of he invention is intended to be understood as including combinations of the various elements of the invention, and of their disclosed or suggested alternatives, including alternatives disclosed, implied or suggested in any one or more of the various methods, products, compositions, systems, apparatus, instruments, aspects, embodiments, examples described in the specification or drawings, if any, and to include any other written or illustrated combination or grouping of elements of the invention or of the possible practice of the invention, except for groups or combinations of elements that will be or become apparent to a person of ordinary skill in the art as being incompatible with or contrary to the purposes of the invention.

Throughout the description, where processes are described as having, including, or comprising specific process steps, it is contemplated that the processes of the invention can also consist essentially of, or consist of, the recited processing steps. It should be understood that the order of steps or order for performing certain actions is immaterial so long as the invention remains operable. Moreover, two or more steps or actions may be conducted simultaneously.

While illustrative embodiments of the invention have been described above, it is, of course, understood that many and various modifications will be apparent to those of ordinary skill in the relevant art, or may become apparent as the art develops, in the light of the foregoing description. Such modifications are contemplated as being within the spirit and scope of the invention or inventions disclosed in this specification.

1. A process for diminishing an undesired endoluminal structure present at a treatment site in a mammalian treatment subject, the endoluminal structure comprising a vascular occlusion, a biofilm or another undesired biological structure and the process comprising:
applying mechanical shockwaves to the endoluminal structure; and
the endoluminal structure absorbing the applied mechanical shockwaves and becoming diminished, dispersed or weakened.

2. A process according to claim 1 comprising delivering a shockwave generating device through a lumen of the mammalian treatment subject to address the treatment site.

3. A process according to claim 1 wherein diminishing the endoluminal structure comprises reducing the mass of, disrupting, attenuating or destroying the endoluminal structure.

4. A process according to claim 1 wherein applying the mechanical shockwaves to the endoluminal structure comprises causing one or more pieces of the endoluminal structure to tear away from the residual endoluminal structure or from the treatment site.

5. A process according to claim 1 wherein applying the mechanical shockwaves comprises impinging a pulsed laser beam on to an ionizable target to generate mechanical shockwaves.

6. A process according to claim 1 wherein applying the mechanical shockwaves comprises impinging a pulsed laser beam on to an ionizable target to form a plasma adjacent the metallic target and to generate mechanical shockwaves emanating from the plasma and moving away from the ionizable target.

7. A process according to claim 6 wherein applying the mechanical shockwaves comprises generating the mechanical shockwaves as non-convergent mechanical shockwaves and directing the non-convergent mechanical shockwaves on to the endoluminal structure resident at the treatment site.

8. A process according to claim 7 comprising employing a treatment instrument to apply the mechanical shockwaves, the treatment instrument including an optical fiber to deliver the laser beam to the metallic target and a distal tip assembly, wherein the distal tip assembly embodies the metallic target and the plasma is formed at the distal tip assembly, the process further comprising inserting the distal tip assembly of the treatment instrument into the mammalian body and applying the mechanical shockwaves while the distal tip is inserted into the mammalian body.

9. A process according to claim 8 comprising manipulating the treatment instrument and directing the mechanical shockwaves on to the endoluminal structure resident at the treatment site.

10. A process according to claim 8 wherein employing a treatment instrument to apply the mechanical shockwaves comprises translating the treatment instrument across the endoluminal structure to incrementally remove material from the endoluminal structure, the treatment instrument optionally being translated across the endoluminal structure in multiple passes.

11. A process according to claim 10 wherein the distal tip comprises a nose portion embodying the metallic target and the process comprises:
the nose portion of the distal tip shielding treatment subject structure from impact with the laser beam; and
the nose portion of the distal tip outputting shockwaves through an output port in a first direction transverse to the optical fiber; and
the nose portion preventing outputting of shockwaves in a direction opposite to the first direction.

12. A process according to claim 10 comprising impacting the mechanical shockwaves on the endoluminal structure laterally of the nose portion of the treatment instrument.

13. A process according to claim 1 comprising advancing the treatment instrument toward or into the endoluminal structure after the removal of material from the endoluminal structure and translating the treatment instrument across the endoluminal structure to incrementally remove additional material from the endoluminal structure.

14. A process according to claim 1 comprising translating, rotating, reciprocating or otherwise moving or manipulating the treatment instrument in relation to the endoluminal structure to incrementally reduce, ablate, disrupt, disperse or weaken endoluminal structure.

15. A process according to claim 1 performed to reduce or weaken a vascular occlusion or constriction.

16. A process according to claim 1 comprising introducing a shockwave generating device into a lumen of the mammalian treatment subject with an introducer, flexing the shockwave-generating device and advancing the shockwave generating device to the treatment site around at least one curve or bend in the mammalian lumen.

17. A process according to claim 1 comprising employing a flexible treatment instrument and a catheter or trocar and inserting the treatment instrument into the vascular system, using the catheter or trocar.

18. A process according to claim 1, wherein the endoluminal structure obstructs a biological fluid flow path in the mammalian treatment subject.

19. A process according to claim 10 wherein the treatment instrument comprises a distal port and wherein the process comprises applying the mechanical shockwaves through the distal port, manipulating the treatment instrument to position the distal port at a distance from the endoluminal structure at the treatment site in the range of from about 0.5 mm to about 10 mm and effecting the applying of mechanical shockwaves with the distal port at said distance from the endoluminal structure.

20. A process according to claim 1 wherein the treatment site is a non-ophthalmologic site and the process comprises controlling the endoluminal structure non-thermolytically or by avoiding delivery of heat to the treatment site or without applying stain to the endoluminal structure or according to a combination of two or all of the foregoing parameters.

21. A process according to claim 1 wherein the endoluminal structure comprises plaque, an embolism, a thrombus, a biofilm or a prophylactic or prosthetic plug.

22. A process according to claim 1 wherein the treatment site comprises a treatment site at a location in the body of the mammalian treatment subject, the location being selected from the group consisting of a coronary artery, a peripheral artery, an otolaryngological site, a nasal, sinus, or middle ear cavity, an implant site, a cardiac implant site, an endovascular implant site, a shunt, an orthopedic implant site, a gynecological implant site, an intrauterine device site, an urologic implant site and a urinary catheter site.

23. A process according to claim 1 comprising controlling the application of mechanical shockwaves to maintain treatment subject tissue at the treatment site intact or free of symptoms of tissue damage or both intact and free of symptoms of tissue damage.

24. A process according to claim 5 wherein applying mechanical shockwaves comprises controlling the application of mechanical shockwaves to the endoluminal structure by selection of one or more control parameters selected from the group consisting of laser energy pulse width, pulse repetition rate, pulse energy and total energy delivered to the target site, the distance of the output port from the target site and the fiber-to-target distance.

25. A process according to claim 5 wherein applying mechanical shockwaves comprises pulsing laser energy impinged on the target to have one or more pulse characteristics selected from the group consisting of a pulse width in the range of from about 2 ns to about 20 ns, a pulse rate of from about 0.5 Hz to about 200 Hz, a pulse energy in a range of from about 2 mJ to about 15 mJ of energy per pulse, and a fiber-to-target distance in the range of from about 0.7 to about 1.5 mm.

26. A process according to claim 5 wherein applying mechanical shockwaves comprises pulsing laser energy impinged on the target to have a pulse width in the range of from about 2 ns to about 20 ns, a pulse rate of from about 0.5 Hz to about 200 Hz, a pulse energy in a range of from about 2 mJ to about 15 mJ of energy per pulse and a fiber-to-target distance in the range of from about 0.7 to about 1.5 mm.

27. A process according to claim 5 to wherein applying mechanical shockwaves comprises impinging pulsed laser energy from an optical fiber on to an ionizable target, wherein the target comprises a metallic structure or material supported independently from the optical fiber, optionally comprises one or more metallic particles.

28. A treatment instrument for controlling an undesired endoluminal structure resident at a treatment site in or on a mammalian treatment subject, wherein the treatment instrument comprises a mechanical shockwave generating assembly configured to apply mechanical shockwaves to the treatment site to control and optionally diminish or weaken the endoluminal structure.

29. A treatment instrument according to claim 28 wherein the mechanical shockwave generating assembly comprises a tip assembly, the tip assembly comprising a short rigid portion connected to a flexible portion and is configured for delivery to a treatment site through a curved lumen of the mammalian treatment subject.

30. A treatment instrument according to claim 29 comprising a flexible outer tube to extend from the treatment site to a location external to the mammalian treatment subject, a short rigid stabilizer tube located within the distal end of the outer tube and a tubular metal tip extending over the distal end of the outer tube and over the stabilizer tube.

31. A treatment instrument according to claim 30 wherein the tubular metal tip comprises a distal nose and the distal nose comprises an ionizable target for transducing laser energy into mechanical shockwaves and an optical fiber extending along the treatment instrument and having a distal end positioned adjacent the ionizable target, the optical fiber being connectable with a pulsed laser energy source to receive pulses of laser energy from the laser energy source and discharge the pulses of laser energy from the distal end of the optical fiber to impinge on the ionizable target, outputting mechanical shockwaves.

32. A treatment instrument according to claim 31 configured for outputting mechanical shockwaves in a mechanical shockwave pattern extending distally and laterally of the treatment instrument to facilitate directing the mechanical shockwaves toward the treatment site.

33. A treatment instrument according to claim 32 wherein the tip assembly comprises a self-supporting unit containable within a small circular cross-section accommodatable in, and moveable along a mammalian lumen to the treatment site.

34. A treatment instrument according to claim 33 wherein the assembly can fit within a containing cross-sectional circle which is less than about 5 mm in diameter and wherein the containing cross-sectional circle optionally is in a range of from about 2 to about 3 mm in diameter.

35. A treatment instrument according to claim 30 wherein the longest rigid, or non-flexible, extent of the tip assembly is less than a distance selected from the group consisting of 20 mm, 15 mm, 10 mm or 8 mm.

36. A treatment instrument according to claim 30 wherein the stabilizer tube has a length in a range of from about 4 mm to about 18 mm or from about 4 mm to about 12 mm or from about 4 mm to about 7 mm.

37. A treatment instrument according to claim 31 wherein the distal nose has a rounded peak located centrally approximately on a central axis of the outer tube.

38. A treatment instrument according to claim 30 capable of impinging pulsed laser energy on the endoluminal structure, the pulsed laser energy having one or more pulse characteristics selected from the group consisting of a pulse width in the range of from about 2 ns to about 20 ns, a pulse rate of from about 0.5 Hz to about 200 Hz, a pulse energy in a range of from about 2 mJ to about 15 mJ of energy per pulse.

39. A treatment instrument according to claim 28 disposed in a bodily cavity of the mammalian subject or housed by a catheter and disposed subcutaneously in the mammalian subject, the treatment instrument having a mechanical shockwave output location disposed adjacent the endoluminal structure.

40. A treatment instrument according to claim 28 comprising a lightly protected optical fiber and metal powder target material.
